(19) United States
(12) Patent Application Publication
(10) Pub. No.: US 2017/0087523 A1
(43) Pub. Date: Mar. 30, 2017

(54) FOAM PRODUCTION EQUIPMENT AND FOAM

(71) Applicant: Eric Poulnais, Le Chesnay (FR)

(72) Inventor: Eric Poulnais, Le Chesnay (FR)

(21) Appl. No.: 15/311,168

(22) PCT Filed: May 13, 2015
(86) PCT No.: PCT/EP2015/060685

§ 371 Date: Nov. 14, 2016

(30) Foreign Application Priority Data
May 16, 2014 (FR) 1454412

(51) Int. Cl. B01F 3/04; B01F 15/02; F41H 9/06; C09K 11/56; B05B 7/00; F41H 9/04; B01F 5/06; A61K 9/12

(52) U.S. Cl. CPC B01F 3/04992; B01F 3/04446; B01F 5/0688; B01F 5/0683; B01F 15/0222; A61K 9/12; C09K 11/565; B05B 7/0037; B05B 7/0075; B05B 7/0081; F41H 9/04; F41H 9/06; B01F 2015/0221; B01F 2215/005

(57) ABSTRACT

The invention relates to foam production equipment comprising:—a first module (A) comprising a principal outlet and delivering at said principal outlet a mixture comprising a principal liquid and at least one surfactant,—a second module (B) coupled to the principal outlet of the first module (A) and producing a foam which comprises at least the principal liquid, the at least one surfactant and at least one principal active product, from the mixture delivered by the principal outlet of the first module (A), the mixture comprising the at least one principal active product or the second module (B) comprising, in addition, means configured such that the foam produced by the second module (B) comprises the at least one principal active product, characterized in that said at least one principal active product is chosen from dyes, fluorescent products, chemical and/or biological labels, irritant products, gases, products which destroy the muscle and/or thinking capacities of a living being and radar reflectors. The invention also relates to a foam which can be produced by such equipment and which advantageously has an expansion coefficient greater than 10.

The invention relates to an apparatus for producing foam containing an additive, for the non-permanent protection against external attacks of a sensitive or defined area or element and/or the marking and/or the camouflaging of an area of interest.

More specifically, the invention relates to an apparatus that makes it possible to produce a foam, this foam occupies a defined volume and has a function of delivering elements that have particular properties.

In the security field, the techniques currently known for the non-permanent protection of a sensitive space are the use of human means and/or the use of irritant gases and/or water cannons and/or smoke grenades.

However, these techniques have several drawbacks.

Protection provided by human means poses the problem of the safety of these same people and of the immediate availability, in the event of a crisis situation, of sufficient personnel in order to ensure the security of the area to be protected.

Protection via the use of water cannons does not allow continuous protection and requires very large volumes of water.

Finally, the use of smoke grenades, of “laden” mist, such as the use of tear gases offers only limited protection over time and also a lack of control of the volume to be protected, due to the rapid dilution of the gases.

Furthermore, smoke grenades are ineffective in the presence of wind, in particular on board a ship.

Thus, there is a need to develop new techniques for protecting sensitive areas that do not have the abovementioned drawbacks.

In the field of marking sensitive areas, in particular in the case of rescue operations, the areas are generally marked by means of colored smoke grenades which can be detected by aircraft.

However, the smoke grenades used disperse rapidly. Furthermore, the area can only be detected visually.

In the field of camouflaging sensitive areas, these areas are generally camouflaged by means of apparatus such as nets or smokescreens.

However, these apparatuses have the drawback of being difficult to use.

Thus, there is a need to develop new methods for marking and/or camouflaging areas that do not have the abovementioned drawbacks.

In particular, in the field of camouflage, these methods should make it possible to make individuals or objects less visible to the eye and/or to visible and/or infrared spectrum cameras and/or to radar.

There is also a need to develop new methods for marking an individual, for example with a biological or biochemical marker of natural or synthetic DNA type. The objective of this generally transparent, odorless and harmless marking is to be able to then remain on the skin for a duration of at least six months and not to be washed off.

Certain apparatuses consist in spraying a mist of active products all around the ship or facility.

However, these apparatuses have the drawback of having to be permanently in operation in order to obtain a high level of protection. Indeed, the mist, once sprayed, dissipates rapidly and is no longer efficient for providing protection.

Thus, there is a need to provide new apparatuses that make it possible to ensure an improved protection and/or marking and/or camouflaging of sensitive areas, for a long duration without needing to operate the apparatus continuously.

Furthermore, the apparatus must be able to be activated rapidly.

Finally, in certain cases, it may be preferable for the apparatus to be portable in order to be able to be used during an intervention.

The applicant has now discovered that an apparatus for producing foam comprising one or more particular additives made it possible to satisfy the abovementioned drawbacks.

One subject of the invention is therefore an apparatus for producing foam comprising:
a first module comprising a main outlet and delivering at said main outlet a mixture comprising a main liquid and at least one surfactant,
a second module coupled to the main outlet of the first module and producing a foam that comprises at least the main liquid, the at least one surfactant and at least one main active product, from the mixture delivered by the main outlet of the first module,

the mixture comprising the at least one main active product or

the second module additionally comprising means configured so that the foam produced by the second module comprises the at least one main active product, characterized in that said at least one main active product is selected from dyes, fluorescent products, chemical and/or biological markers, irritant products, gases, products that destroy the muscular and/or thought capacities of a living being and radar reflectors.

The apparatus according to the invention is advantageously configured for producing a foam with a high expansion coefficient, that is to say greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

This apparatus therefore makes it possible to produce a foam with such expansion coefficients.

Another subject of the invention is a foam capable of being produced by the apparatus described above and advantageously having an expansion coefficient greater than 10, very advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

The foam according to the invention may be sprayed from far away, for example from 2 to 15 meters or even more than 50 meters or on the contrary be used locally at the production site of this foam.

The expansion coefficient may vary according to the amount of gas introduced into the mixture but may also depend on parameters such as the choice of the surfactant, the concentration of this agent, the type of nozzle used when nozzles are used and the mesh of the grid if a grid is used.

The term “gas” is understood in the present invention to mean any gas conventionally used in devices for producing foam.

Preferably, in the present invention, the gas is air.

The foam according to the invention may be invasive and/or have a good hold and/or be very sticky and enable a vast space to be occupied.

The foam according to the invention may be used both in a closed space and in an open area.

Furthermore, the foam remains correctly located in the area to be protected and/or to be marked and/or to be camouflaged.

Furthermore, it generally withstands the action of the wind.

Furthermore, it visually delimits the areas where the main active product(s) are spread, unlike a laden gas or mist.

Furthermore, by means of the main active product(s) that it contains, it enables the protection and/or the marking and/or the camouflaging (additional to the simple visualization of the foam) of a sensitive area.

Advantageously, in the case of an area to be protected, the area may be rendered inaccessible to people not protected with special equipment.

Finally, this foam may be durable over time and be stable from several minutes to several hours or several tens of hours, depending on the nature and the content of the chosen surfactant(s).

The apparatus according to the invention may be used both outside and inside a building and also on or in a vehicle.

Consequently, another subject of the invention is a motor vehicle, ship or aircraft comprising the apparatus as described above.

A final subject of the invention is a process for producing foam having an expansion coefficient of greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

In the present description, the expansion coefficient is conventionally defined as being the ratio of the volume of foam produced to the volume of the mixture used to form this foam.

One subject of the invention is therefore an apparatus for producing foam comprising:
a first module comprising a main outlet and delivering at said main outlet a mixture comprising a main liquid and at least one surfactant,
a second module coupled to the main outlet of the first module and producing a foam that comprises at least the main liquid, the at least one surfactant and at least one main active product, from the mixture delivered by the main outlet of the first module,

the mixture comprising the at least one main active product or

the second module additionally comprising means configured so that the foam produced by the second module comprises the at least one main active product, characterized in that said at least one main active product is selected from dyes, fluorescent products, chemical and/or biological markers, irritant products, gases, products that destroy the muscular and/or thought capacities of a living being and radar reflectors.

Advantageously, the apparatus produces a foam that has an expansion coefficient of greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

The choice of the desired expansion coefficient depends on the mode of use of the apparatus, on the site for generating the foam and on its composition.

The apparatus according to the invention has several modes of implementation for each of the two variants of the apparatus.

In a first variant of the apparatus according to the invention, the mixture delivered by the main outlet of the first module comprises the at least one main active product.

In this first variant, the first module of the apparatus according to the invention may have various modes of implementation.

In a first mode of implementation of this first module, it comprises a single reservoir which comprises the main liquid, the at least one surfactant and the at least one main active product, this reservoir being connected to the main outlet of the first module.

This “all-in-one” mode of implementation may in particular be used in a “portable” mode of the apparatus according to the invention.

In a second mode of implementation of this first module, it comprises a main reservoir which comprises the main liquid, said main reservoir being connected to a main inlet of a main pipe, and a first reservoir which comprises the at least one surfactant and the at least one main active product, said first reservoir being in fluidic communication with a first secondary inlet of the main pipe, said main pipe being connected to the main outlet of the first module.

In a third mode of implementation of this first module, it comprises a main reservoir which comprises the main liquid and the at least one surfactant, said main reservoir being connected to a main inlet of a main pipe, and a first reservoir which comprises the at least one main active product, said first reservoir being in fluidic communication with a first secondary inlet of the main pipe, said main pipe being connected to the main outlet of the first module.

In a fourth mode of implementation of this first module, it comprises a main reservoir which comprises the main liquid and the at least one main active product, said main reservoir being connected to a main inlet of a main pipe, and a first reservoir which comprises the at least one surfactant, said first reservoir being in fluidic communication with a first secondary inlet of the main pipe, said main pipe being connected to the main outlet of the first module.

In a fifth mode of implementation of this first module, it comprises a main reservoir which comprises the main liquid, said main reservoir being connected to a main inlet of a main pipe, a first reservoir which comprises the at least one surfactant, said first reservoir being in fluidic communication with a first secondary inlet of the main pipe, and a second reservoir which comprises the at least one main active product, said second reservoir being in fluidic communication with a second secondary inlet of the main pipe, said main pipe being connected to the main outlet of the first module.

In a sixth mode of implementation of this first module, it comprises a main inlet of a main pipe connected to an intake of main liquid, a first reservoir which comprises the at least one surfactant and the at least one main active product, said first reservoir being in fluidic communication with a first secondary inlet of the main pipe, said main pipe being connected to the main outlet of the first module.

In a seventh mode of implementation of this first module, it comprises a main inlet of a main pipe connected to an intake of main liquid, a first reservoir which comprises the at least one surfactant, said first reservoir being in fluidic communication with a first secondary inlet of the main pipe, and a second reservoir which comprises the at least one main active product, said second reservoir being in fluidic communication with a second secondary inlet of the main pipe, said main pipe being connected to the main outlet of the first module.

In a second variant of the apparatus according to the invention, the second module also comprises means configured so that the foam produced by the second module comprises the at least one main active product.

In this second variant, the first module of the apparatus according to the invention may comprise various modes of implementation.

In a first mode of implementation of this first module, it comprises a single reservoir which comprises the main liquid, the at least one surfactant and optionally at least one secondary active product, this reservoir being connected to the main outlet of the first module.

This “all-in-one” mode of implementation may in particular be used in a “portable” mode of the apparatus according to the invention.

In a second mode of implementation of this first module, it comprises a main reservoir which comprises the main liquid, and optionally at least one secondary active product, said main reservoir being connected to a main inlet of a main pipe, and a first reservoir which comprises the at least one surfactant, said first reservoir being in fluidic communication with a first secondary inlet of the main pipe, said main pipe being connected to the main outlet of the first module.

In a third mode of implementation of this first module, it comprises a main reservoir which comprises the main liquid, said main reservoir being connected to a main inlet of a main pipe, and a first reservoir which comprises the at least one surfactant and optionally at least one secondary active product, said first reservoir being in fluidic communication with a first secondary inlet of the main pipe, said main pipe being connected to the main outlet of the first module.

In a fourth mode of implementation of this first module, it comprises a main reservoir which comprises the main liquid and the at least one surfactant, said main reservoir being connected to a main inlet of a main pipe, and a first reservoir which comprises at least one secondary active product, said first reservoir being in fluidic communication with a first secondary inlet of the main pipe, said main pipe being connected to the main outlet of the first module.

In a fifth mode of implementation of this first module, it comprises a main reservoir which comprises the main liquid, said main reservoir being connected to a main inlet of a main pipe, a first reservoir which comprises the at least one surfactant, said first reservoir being in fluidic communication with a first secondary inlet of the main pipe, and a second reservoir which comprises at least one secondary active product, said second reservoir being in fluidic communication with a second secondary inlet of the main pipe, said main pipe being connected to the main outlet of the first module.

In a sixth mode of implementation of this first module, it comprises a main inlet of a main pipe connected to an intake of main liquid, a first reservoir which comprises the at least one surfactant and optionally at least one secondary active product, said first reservoir being in fluidic communication with a first secondary inlet of the main pipe, said main pipe being connected to the main outlet of the first module.

In a seventh mode of implementation of this first module, it comprises a main inlet of a main pipe connected to an intake of main liquid, a first reservoir which comprises the at least one surfactant, said first reservoir being in fluidic communication with a first secondary inlet of the main pipe, and a second reservoir which comprises at least one secondary active product, said second reservoir being in fluidic communication with a second secondary inlet of the main pipe, said main pipe being connected to the main outlet of the first module.

Both in the first variant and in the second variant of this apparatus, the single reservoir of the first module or the main reservoir of the first module may preferably have an inlet connected to a pyrotechnic system, or to a compressed gas storage and/or production system, in order to pressurize the apparatus.

Finally, whether in the first variant or in the second variant of this apparatus, the single reservoir of the first module or the main reservoir of the first module may be under a pressure greater than the atmospheric pressure.

Preferably, the compressed gas storage and/or production system comprises a compressor and/or a pressurized gas cylinder.

Advantageously, the second module of the apparatus according to the invention is configured for producing a foam with an expansion coefficient greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

The second module of the apparatus according to the invention may have various modes of implementation.

In a first mode of implementation of this second module, it comprises a main inlet connected to the main outlet of the first module, said main inlet being connected to at least one main nozzle, a grid being positioned downstream of said at least one main nozzle, and, upstream of said at least one main nozzle, means configured for producing a blast in the flow direction of the mixture present at the main inlet of the second module.

Within the meaning of the present invention the term “blast” is understood to mean a stream of gas.

In a second mode of implementation of this second module, it comprises a main inlet connected to the main outlet of the first module, said main inlet being connected to at least one main nozzle, a gas inlet area in the form of a housing being positioned downstream of said at least one main nozzle, the longitudinal axis of this inlet area being parallel or identical to the spraying axis of said at least one main nozzle, and downstream of this inlet area, a grid positioned over the outlet of the inlet area.

Preferably, the housing may be annular.

In this embodiment, the grid may be of frustoconical shape, in the form of a spherical surface, in the form of an elliptical surface, of flat shape or of any intermediate shape.

In a third mode of implementation of this second module, it comprises a main inlet connected to the main outlet of the first module, said main inlet being connected to a final reservoir, said final reservoir comprising a sparging system.

Preferably, the sparging system in the second module of the apparatus according to the invention is uniformly distributed over the entire surface of the bottom of the final reservoir.

Advantageously, a grid may be located above the final reservoir or at the outlet of a pipe channeling the foam.

The sparging system may advantageously comprise an inlet, said inlet being connected to a pyrotechnic system, or to a compressed gas storage and/or production system as defined above.

The sparging system may also advantageously be connected to a reservoir under a pressure greater than the atmospheric pressure.

In the first and second mode of implementation of this second module, the mesh of the grid is not limiting in the apparatus according to the invention.

The choice of the grid mesh size may depend in particular on the desired size of the bubbles, on the velocity of the gas flow that will form the bubbles of foam, on the nature of the surfactant and on the viscosity of the main mixture.

However, generally, the mesh will preferably be between 1 and 4 mm in diameter. It may also be produced with a honeycomb.

In the first and the second mode of implementation of this second module, the means configured for producing a blast are generally configured for producing a foam with an expansion coefficient of greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

In the first and the second mode of implementation of this second module, the means configured for producing a blast generally comprise a fan or a pyrotechnic system.

In the apparatus according to the invention, the pyrotechnic system may be any device conventionally used for the rapid triggering of a blast.

In the apparatus according to the invention, the pyrotechnic system generally comprises an explosive composition.

Advantageously, the explosive composition may be any explosive composition known to those skilled in the art.

Advantageously, the main pipe of the first module comprises a valve located downstream of the main inlet of the first module or of the main reservoir of the first module or of the single reservoir of the first module and upstream, if they are present, of the secondary inlet(s).

This or these valve(s) make it possible to control the fluid supply of the apparatus.

In one particular embodiment of the apparatus according to the invention, the foam produced by the second module also comprises at least one additive.

This additive may in particular be a stabilizer enabling a high stability over time to be imparted to the foam.

This additive may be present, for example, in the single reservoir, the main reservoir, the first reservoir and/or the second reservoir, if they are present in the apparatus according to the invention.

Advantageously in this particular embodiment of the apparatus, the first module may comprise at least one third reservoir which comprises the at least one additive, said third reservoir being in fluidic communication with a third secondary inlet of the main pipe of the first module.

Advantageously, the at least one additive may be selected from surfactants different from the at least one surfactant, preferably 1-dodecanol, products that increase the viscosity, preferably albumin, gelatin, and polysaccharides such as carboxymethyl cellulose and gums, in particular gum arabic, foam stabilizers, antifreeze compounds, preserving agents and a mixture of these additives.

The at least one additive may make it possible in particular to stabilize the foam and/or the at least one main active product and optionally the at least one secondary active product.

Advantageously, the mixture delivered at the main outlet of the first module has a content of additive, if it is present, that varies from 0.0001% to 10% by weight, preferably from 0.001% to 1% by weight, relative to the total weight of the mixture delivered at the main outlet of the first module.

The location of the secondary inlets on the main pipe is of no importance in the apparatus as claimed.

For example, the second secondary inlet may just as well be upstream as downstream of the first secondary inlet.

Similarly, the third secondary inlet may very well be located upstream or downstream of the first and second secondary inlets or be located between these two secondary inlets.

Advantageously, the fluidic communications of the reservoirs with the secondary inlets are carried out by means of mixers.

These mixers make it possible to obtain a homogeneous liquid, in the case where the mixture is completely liquid, or a homogeneous distribution of the gas and/or of the solid particles in the liquid in the case where the mixture is heterogeneous.

Advantageously, the apparatus may comprise a control system that controls the operation, when they are present, of the valve and/or of the mixers and/or of the means configured for producing a blast.

The control system may be manual, semiautomatic or automatic.

Preferably, when the control system is manual, it comprises one or more push buttons.

Preferably, when the control system is semiautomatic, it comprises one or more programmable industrial logic controllers, one or more control consoles and one or more contactors enabling the activation of the foam production.

Preferably, the control system may be automatically controlled by a sensing device, a video surveillance system, an infrared detector cell or an infrared camera for example.

Advantageously, the means configured so that the foam produced by the second module comprises the at least one main active product may comprise an addition device which comprises a supplementary reservoir which comprises the at least one main active product, the supplementary reservoir being connected to at least one means capable of adding the at least one main active product to the mixture present at the main inlet of the second module.

In the particular cases where the main inlet of the second module is connected to at least one main nozzle, the means capable of adding the at least one main active product to the mixture present at the main inlet of the second module may comprise at least one supplementary nozzle, the flow of the at least one main active product being in the same direction as the flow of the mixture present at the main inlet of the second module.

This addition device advantageously makes it possible to add to the foam bubbles being formed a gas and/or a liquid in suspension such as a mist, and/or a solid, such as a smoke, trapped inside the bubbles.

Advantageously, the apparatus according to the invention may also comprise one or more additional reservoirs configured to each receive one or more additional additives, said additional reservoir(s) each being in fluidic communication with an additional secondary inlet of the main pipe.

The additional secondary inlet(s) may be placed at any location of the main pipe, for example upstream or downstream of the secondary inlets.

The apparatus according to the invention makes it possible, inter alia, to protect sensitive land-based buildings such as crisis management stations, logistics hubs, data processing centers, prisons, communication centers, all types of nerve centers and landmarks that are vital on a regional and/or national level and/or that are isolated (wind turbines, lighthouses, aircraft, jewelry stores, banks, governmental establishments, etc.).

The apparatus may also protect and be on mobile elements such as ships of all sizes but also floating or stationary structures such as platforms, barges, marine turbines, dams, equipment/food/personnel transport vehicles, VIP vehicles, convoys, vehicles for transporting valuable funds, judicial transfer vehicles, vehicles on wheels, tracks or rails.

The apparatus may also be intended to be borne by a human being.

The apparatus may also be intended to camouflage an object (for example a vehicle) or a human being.

Advantageously, the main liquid of the apparatus according to the invention is selected from a freshwater, a seawater, an oil, an alcohol and/or a mixture of these liquids.

Advantageously, the main liquid intake may comprise a municipal water circuit or a pump that directly withdraws the water from a natural source, such as seawater for example.

Generally, the main liquid intake is connected to a network under a pressure greater than the atmospheric pressure, which enables the displacement of the main liquid in the apparatus.

The single reservoir and the main reservoir of the apparatus according to the invention may advantageously be under a pressure greater than the atmospheric pressure.

This pressure may in particular be obtained by means of a pyrotechnic system, or by means of a compressed gas storage and/or production system as defined above.

Advantageously, the at least one surfactant present in the apparatus according to the invention may be selected from protein-based surfactants obtained by hydrolysis (decomposition) of animal or plant proteins or synthetic-based surfactants derived from the chemical industry or else any molecule capable of generating a foam.

The at least one surfactant will preferably be chosen from ionic surfactants and nonionic surfactants, and preferably from ionic surfactants that have a linear hydrophobic chain such as dodecyl sulfate, with addition of the order of 1% of nonionic co-surfactants such as C₁₂ to C₁₄ linear chain monoethanolamides, or isopropanolamides, or else diethanolamides.

Mixtures of various types of surfactant (anionic, cationic, amphoteric) are possible according to the features of the desired foam (expansion, settling).

The choice of the surfactant may be made on the basis of the desired application for the foam produced.

Advantageously, the content of surfactant in the mixture delivered at the main outlet of the first module varies from 1% to 10% by weight, and preferably from 2% to 7% by weight, relative to the total weight of the mixture present at the main outlet of the main pipe.

In the present application, an active product is understood to mean any product whose main objective is not to modify the physical features of the foam, such as the expansion coefficient, its consistency, its viscosity or its density.

According to a first preferred mode, an active product is understood to mean a compound that impairs the physical capacities of a living being, in particular of a human being.

Advantageously in this first preferred mode, the at least one secondary active product, if it is present, may be selected from irritant products and products that destroy the muscular and/or thought capacities of a living being.

According to a second preferred mode, an active product is understood to mean a compound that imparts a marking to the element with which it is in contact.

Advantageously in this second preferred mode, the at least one secondary active product, if it is present, may be selected from dyes, fluorescent products and chemical and/or biological markers.

According to a third preferred mode, an active product is understood to mean a compound that imparts a camouflaging to a particular area.

Advantageously in this third preferred mode, the at least one secondary active product, if it is present, may be selected from dyes.

According to a fourth preferred mode, an active product is understood to mean a compound selected from gases and radar reflectors.

In the foam production apparatus according to the invention, the at least main active product is selected from dyes, fluorescent products, chemical and/or biological markers, irritant products, gases, products that destroy the muscular and/or thought capacities of a living being and radar reflectors.

As irritant product or product that destroys the muscular and/or thought capacities of a living being, use could in particular be made of capsicum oleoresin (OC) or a derivative thereof, chlorobenzylidene malononitrile (CS), chloroacetophenone (CN), bromobenzyl cyanide (CA), dibenzo(b,f,)-1,4-oxazepine (CR), nonivamide (PAVA), capsaicin, dihydrocapsaicin, nordihydrocapsaicin, homodihydrocapsaicin and homocapsaicin.

In one particular variant of the invention, the irritant product(s) and the product(s) that destroy(s) the muscular and/or thought capacities of a living being may be in a form encapsulated in gum arabic, and in particular capsicum oleoresin or a derivative thereof and nonivamide.

As dye, use could in particular be made of those having a high solubility in the main liquid, preferably water and a high chemical stability.

Methylene blue, eosin Y (red), radamine WT (red), Lissamine falavine FF (yellow) and food colorings such as for example patent blue V [E131], azorubine [E122] (red) and quinoline yellow [E104] (yellow) could in particular be favored.

As fluorescent product, use could in particular be made of zinc sulfide, wurtzite, of base formula ZnS combined with a heavy metal, scheelite (CaWO₄), or another alkali or alkaline-earth metal sulfide such as CaS for example.

As chemical and/or biological marker, use could in particular be made of synthetic DNA composed of a sequence of the four nucleic acids that form it: adenine, thymine, guanine and cytosine.

As radar reflector, use could in particular be made of compounds visible by radars such as fine drops of oil containing very fine metal powder.

Preferably, the at least one main active product and/or the at least one optional secondary active product are selected from irritant products and products that destroy the muscular and/or thought capacities of a living being, and preferably, the at least one main active product and/or the at least one optional secondary active product are capsaicin, capsicum oleoresin or a derivative thereof and nonivamide.

Advantageously, the at least one main active product and the at least one optional secondary active product each have a content that varies from 0.1% to 10% by weight, preferably from 0.5% to 3% by weight, relative to the total weight of the mixture delivered at the main outlet of the first module.

Advantageously, the main active product(s) may have an efficacy duration that is shorter or as long as the lifetime of the foam.

The additional reservoir(s) of the apparatus according to the invention, if they are present therein, each comprise one or more additional additives.

Said additional additive(s) may be selected from those mentioned above as surfactant, main active product and additive.

Advantageously, the supplementary reservoir of the apparatus according to the invention, if it is present, comprises a supplementary fluid selected from a gas, a liquid, optionally with fine particles of active product.

Another subject of the invention is a foam capable of being produced by the apparatus as defined above, advantageously having an expansion coefficient of greater than 10, very advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

Advantageously, the at least one main active product is located in the wall of the bubbles of the foam according to the invention and/or in the form of gas inside the bubbles of the foam and/or in the form of fine liquid or solid particles in suspension inside the bubbles of the foam.

When the main additive is in liquid form, it is located in the wall of the bubbles of the foam.

When the main additive is in the form of gas, or fine liquid or solid particles in suspension, it is located inside the bubbles of the foam.

Another subject of the invention is the use of the apparatus described above for protecting sensitive land-based buildings such as crisis management stations, logistics hubs, data processing centers, prisons, communication centers, all types of nerve centers and landmarks that are vital on a regional and/or national level and/or that are isolated (wind turbines, lighthouses, aircraft, jewelry stores, banks, governmental establishments, etc.).

The apparatus may also be used to protect and be on mobile elements such as ships of all sizes but also floating or stationary structures such as platforms, barges, marine turbines, dams, equipment/food/personnel transport vehicles, VIP vehicles, convoys, vehicles for transporting valuable funds, judicial transfer vehicles, vehicles on wheels, tracks or rails.

The apparatus may also be intended to be borne by a human being.

The apparatus may also be intended to camouflage an object (for example a vehicle) or a human being.

Another subject of the invention is a motor vehicle, ship or aircraft comprising the apparatus as defined above.

A final subject of the invention is a process for producing a foam comprising the following steps:

i) a first module that delivers at the main outlet a mixture comprising a main liquid and at least one surfactant, then

ii) a second module connected to the main outlet of the first module that produces a foam which comprises the main liquid, the at least one surfactant and at least one main active product, it being understood that the foam produced has an expansion coefficient of greater than 10, advantageously greater than 20, preferably varying from 20 to 2000, more preferentially varying from 40 to 2000, more preferentially still varying from 60 to 2000, more preferentially still varying from 150 to 2000, and in particular varying from 400 to 2000.

Other advantages and features of the invention will appear more clearly on examining the detailed description of the various modes of implementation of the invention, which are in no way limiting, and of the appended drawings, in which:

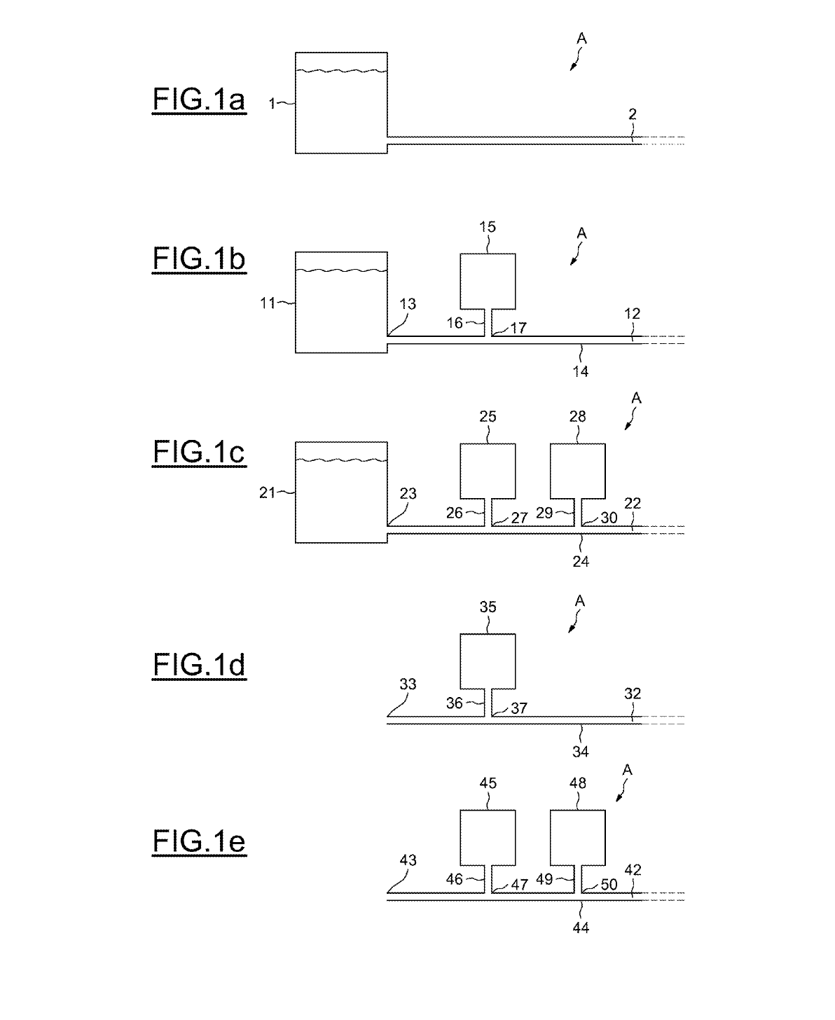

FIGS. 1a to 1e schematically present various embodiments of the first module;

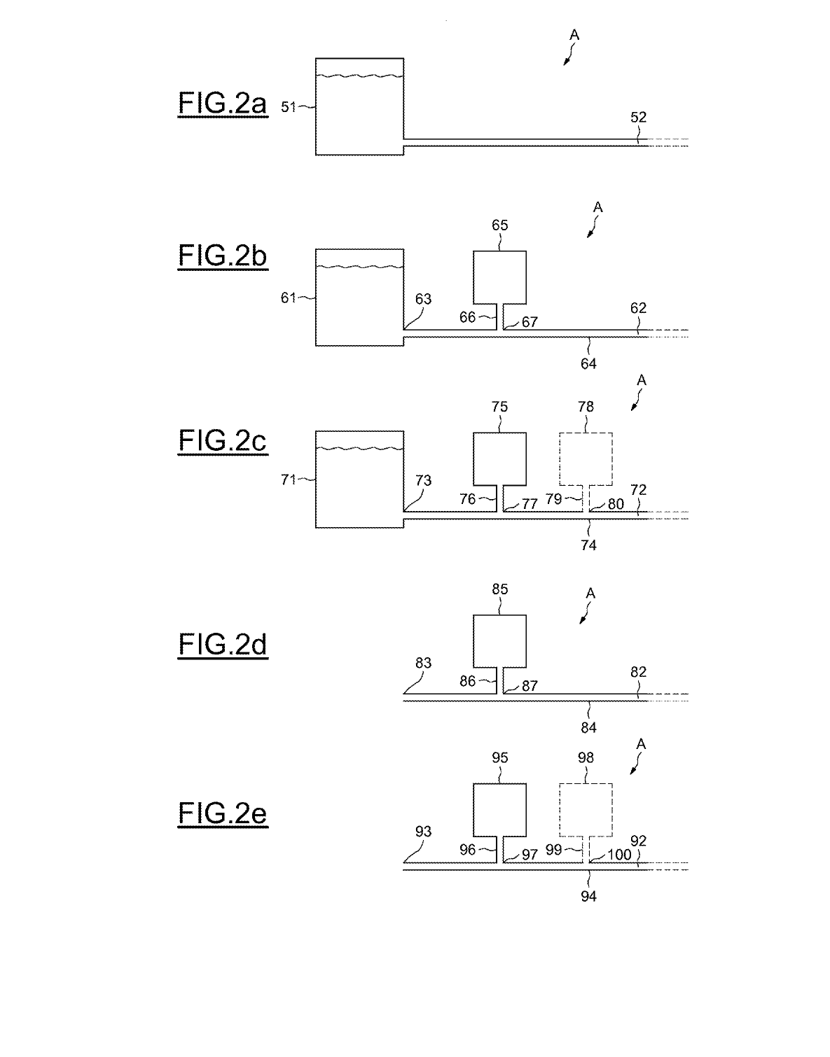

FIGS. 2a to 2e schematically present various embodiments of the first module;

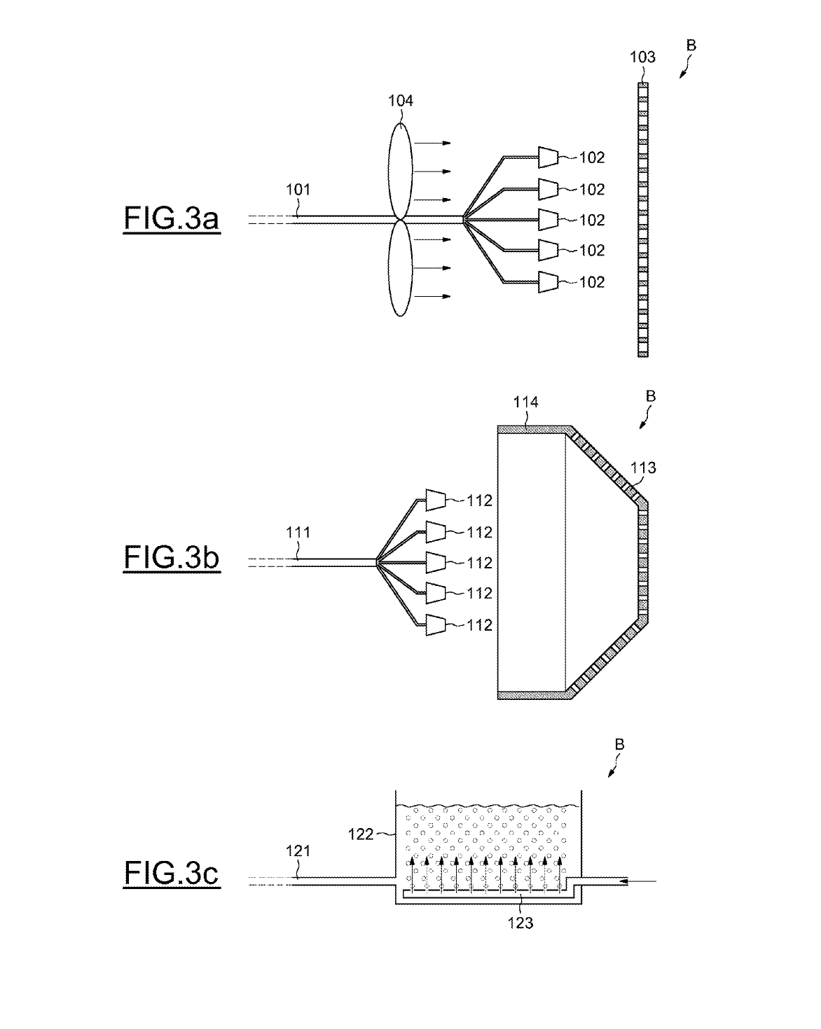

FIGS. 3a to 3c schematically present various embodiments of the second module;

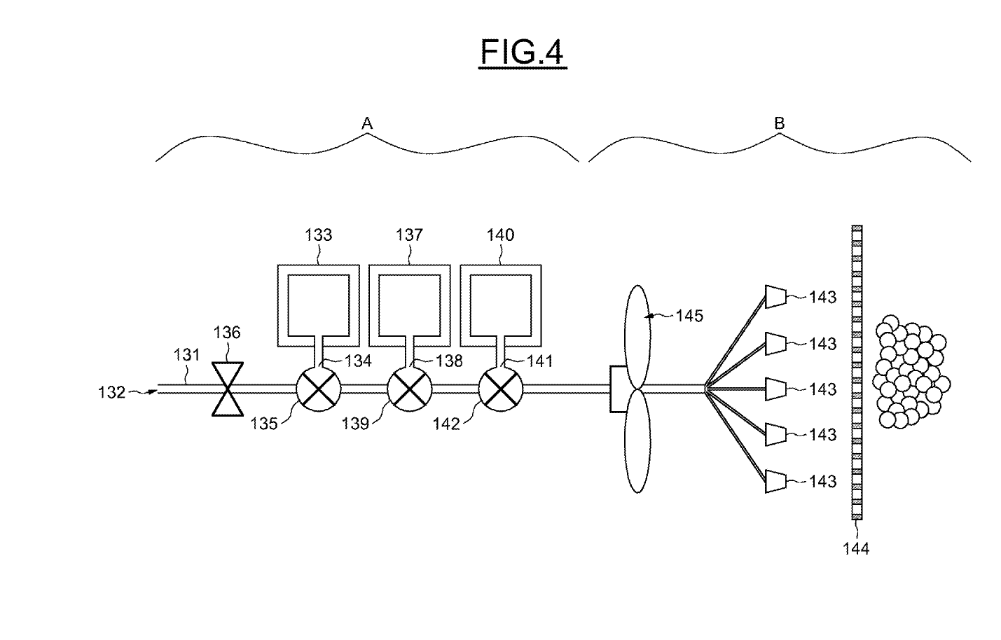

FIG. 4 schematically presents an apparatus for producing foam according to a first embodiment;

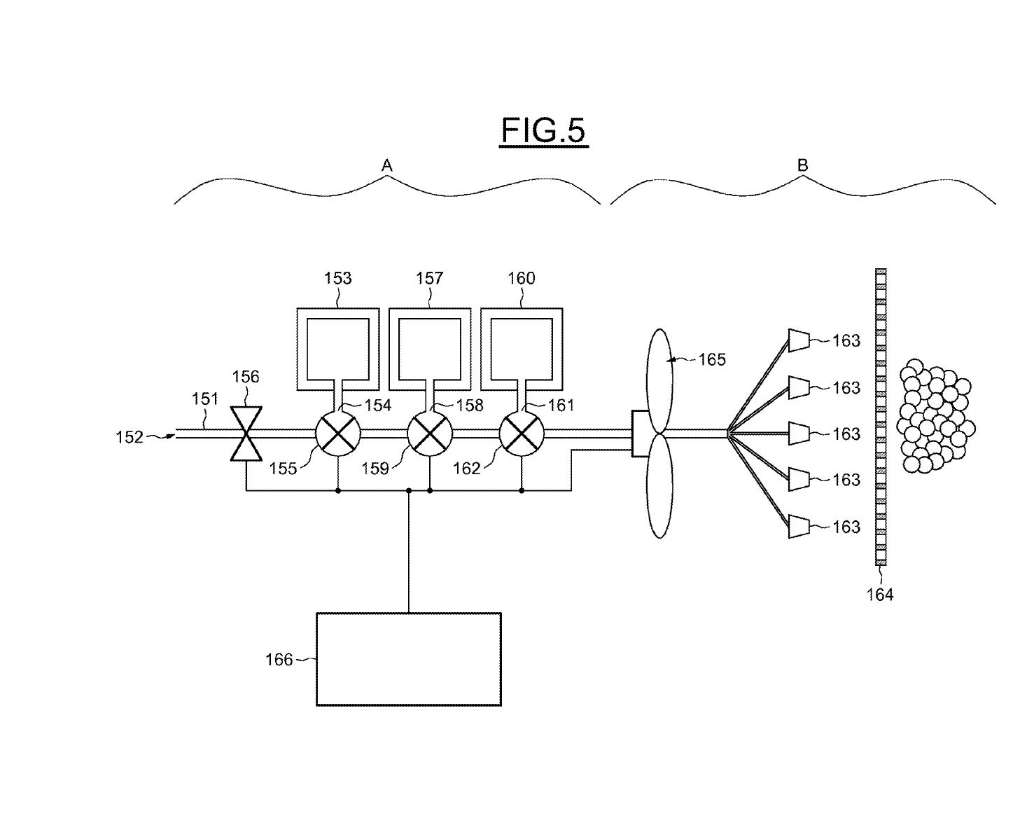

FIG. 5 schematically presents an apparatus for producing foam according to a second embodiment;

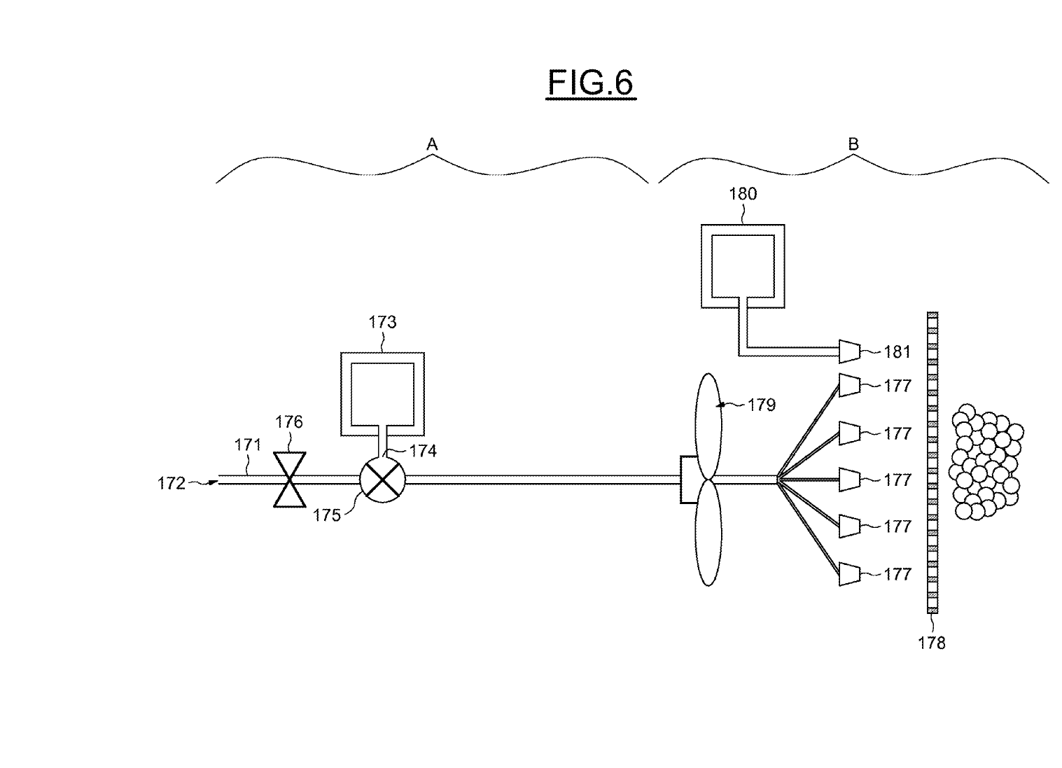

FIG. 6 schematically presents an apparatus for producing foam according to a third embodiment;

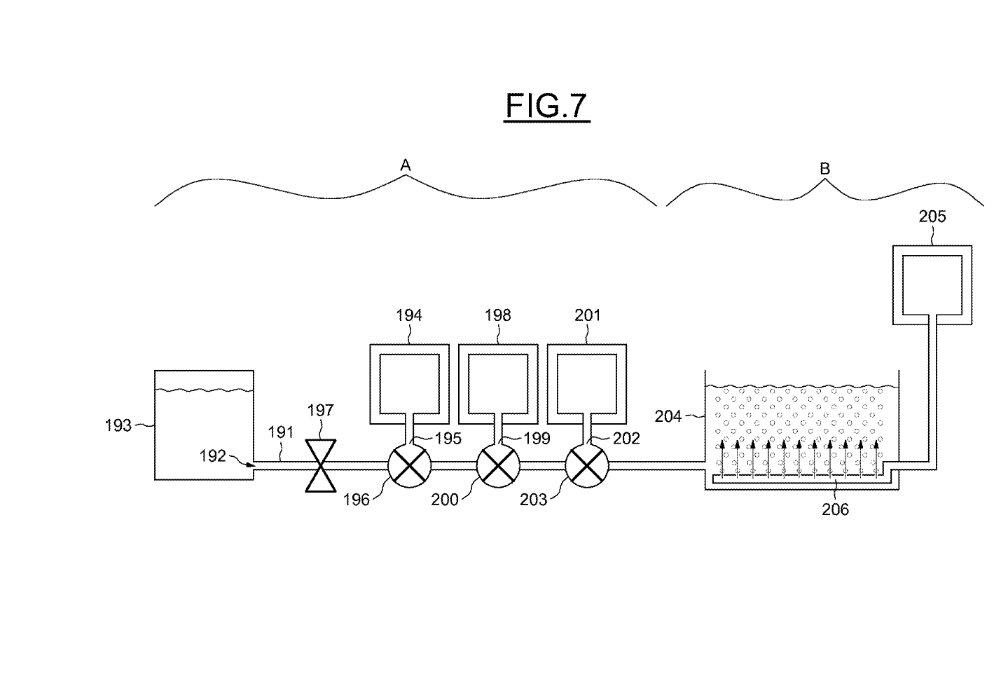

FIG. 7 schematically presents an apparatus for producing foam according to a fourth embodiment;

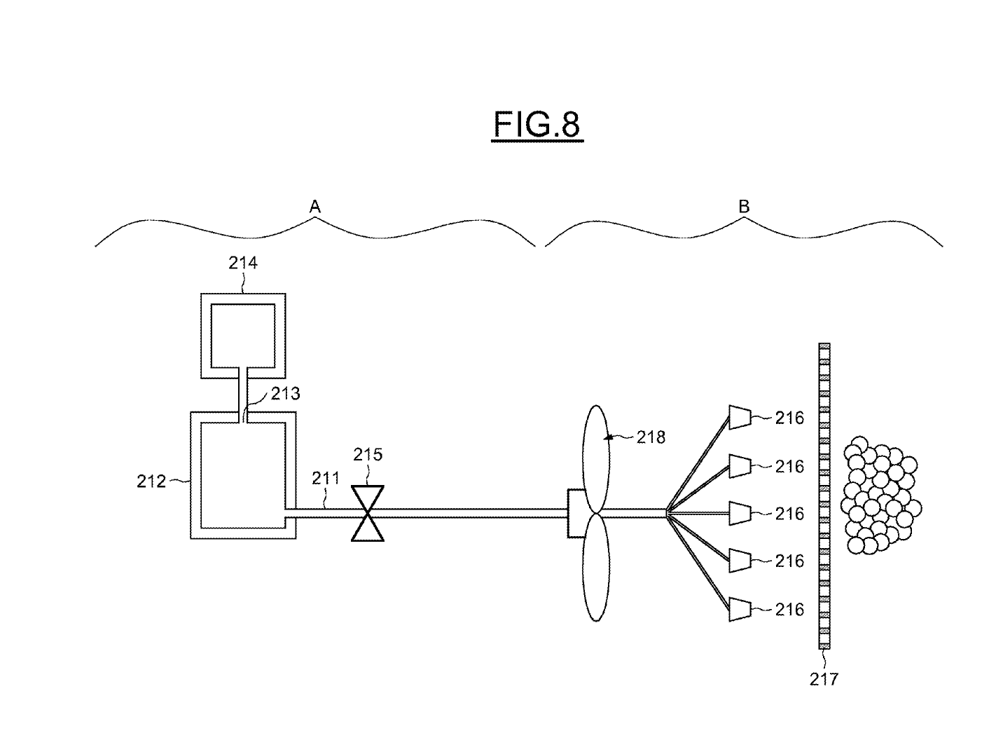

FIG. 8 schematically presents an apparatus for producing foam according to a fifth embodiment;

FIG. 9 schematically presents an apparatus for producing foam according to a fifth embodiment.

FIGS. 1a to 1e describe the various modes of implementation of a first module A in the first variant of the apparatus according to the invention.

In FIG. 1a, the first module comprises a single reservoir 1 which comprises the main liquid, the at least one surfactant and the at least one main active product, this reservoir 1 being connected to the main outlet 2 of the first module.

In FIG. 1b, the first module comprises a main reservoir 11 which comprises the main liquid, and optionally the at least one surfactant or the at least one main active product, said main reservoir 11 being connected to a main inlet 13 of a main pipe 14, and a first reservoir 15 which comprises the at least one surfactant and/or the at least one main active product, said first reservoir 15 being in fluidic communication 16 with a first secondary inlet 17 of the main pipe 14, said main pipe being connected to the main outlet 12 of the first module.

In FIG. 1c, the first module comprises a main reservoir 21 which comprises the main liquid, said main reservoir 21 being connected to a main inlet 23 of a main pipe 24, a first reservoir 25 which comprises the at least one surfactant, said first reservoir 25 being in fluidic communication 26 with a first secondary inlet 27 of the main pipe 24, and a second reservoir 28 which comprises the at least one main active product, said second reservoir being in fluidic communication 29 with a second secondary inlet 30 of the main pipe 24, said main pipe 24 being connected to the main outlet 22 of the first module.

In FIG. 1d, the first module comprises a main inlet 33 of a main pipe 34 connected to an intake of main liquid, a first reservoir 35 which comprises the at least one surfactant and the at least one main active product, said first reservoir 35 being in fluidic communication 36 with a first secondary inlet 37 of the main pipe 34, said main pipe 34 being connected to the main outlet 32 of the first module.

In FIG. 1e, the first module comprises a main inlet 43 of a main pipe 44 connected to an intake of main liquid, a first reservoir 45 which comprises the at least one surfactant, said first reservoir 45 being in fluidic communication 46 with a first secondary inlet 47 of the main pipe 44, and a second reservoir 48 which comprises the at least one main active product, said second reservoir 48 being in fluidic communication 49 with a second secondary inlet 50 of the main pipe 44, said main pipe 44 being connected to the main outlet 42 of the first module.

FIGS. 2a to 2e depict the various modes of implementation of a first module A in the second variant of the apparatus according to the invention.

In FIG. 2a, the first module comprises a single reservoir 51 which comprises the main liquid, the at least one surfactant and optionally at least one secondary active product, this reservoir 51 being connected to the main outlet 52 of the first module.

In FIG. 2b, the first module comprises a main reservoir 61 which comprises the main liquid, and optionally the at least one surfactant or at least one secondary active product, said main reservoir 61 being connected to a main inlet 63 of a main pipe 64, and a first reservoir 65 which comprises the at least one surfactant and/or optionally the at least one secondary active product, said first reservoir 65 being in fluidic communication 66 with a first secondary inlet 67 of the main pipe 64, said main pipe 64 being connected to the main outlet 62 of the first module.

In FIG. 2c, the first module comprises a main reservoir 71 which comprises the main liquid, said main reservoir being connected to a main inlet 73 of a main pipe 74, a first reservoir 75 which comprises the at least one surfactant, said first reservoir 75 being in fluidic communication 76 with a first secondary inlet 77 of the main pipe 74, and optionally a second reservoir 78 which comprises at least one secondary active product, said second reservoir 78 being in fluidic communication 79 with a second secondary inlet 80 of the main pipe 74, said main pipe 74 being connected to the main outlet 72 of the first module.

In FIG. 2d, the first module comprises a main inlet 83 of a main pipe 84 connected to an intake of main liquid, a first reservoir 85 which comprises the at least one surfactant and optionally at least one secondary active product, said first reservoir 85 being in fluidic communication 86 with a first secondary inlet 87 of the main pipe 84, said main pipe 84 being connected to the main outlet 82 of the first module,

In FIG. 2e, the first module comprises a main inlet 93 of a main pipe 94 connected to an intake of main liquid, a first reservoir 95 which comprises the at least one surfactant, said first reservoir 95 being in fluidic communication 96 with a first secondary inlet 97 of the main pipe, and optionally a second reservoir 98 which comprises at least one secondary active product, said second reservoir 98 being in fluidic communication 99 with a second secondary inlet 100 of the main pipe 94, said main pipe 94 being connected to the main outlet 92 of the first module.

FIGS. 3a to 3c depict various modes of implementation of a second module B.

In FIG. 3a, the second module comprises a main inlet 101 connected to the main outlet of the first module, said main inlet 101 being connected to at least one main nozzle 102, a grid 103 being positioned downstream of said at least one main nozzle 102, and, upstream of said at least one main nozzle 102, means configured for producing a blast 104 in the flow direction of the mixture present at the main inlet 101 of the second module.

In FIG. 3b, the second module comprises a main inlet 111 connected to the main outlet of the first module, said main inlet 111 being connected to at least one main nozzle 112, a gas inlet area 114 in the form of a housing being positioned downstream of said at least one main nozzle 112, the longitudinal axis of this inlet area 114 being parallel or identical to the spraying axis of said at least one main nozzle 112, and downstream of this inlet area, a grid 113 positioned over the outlet of the inlet area 114.

In the case of FIG. 3b, this embodiment of the second module functions as follows: the mixture at the main outlet of the first module arrives at the main inlet of the second module 111 and is sprayed by means of at least one main nozzle 112 across the inlet area 114. This produces a Venturi effect which supplies a gas to the mixture.

Everything is then sprayed onto a grid 113 that makes it possible to produce the foam bubbles.

In FIG. 3c, the second module comprises a main inlet 121 connected to the main outlet of the first module, said main inlet 121 being connected to a final reservoir 122, said final reservoir comprising a sparging system 123.

FIG. 4 presents a first mode of implementation of the foam production apparatus according to the invention.

This apparatus comprises a main pipe 131 having a main inlet 132. This main inlet is connected to an intake of main liquid. The intake of main liquid possibly being as defined above.

The apparatus comprises a first reservoir 133 which comprises the at least one surfactant as defined above.

The first reservoir 133 is in fluidic communication with a first secondary inlet 134 of the main pipe 131.

The fluidic communication is carried out by means of a mixer 135.

Preferably, the mixer 135 operates with a suction system via the Venturi effect or via an injection pump.

Downstream of this first mixer 135, a valve 136 is present on the main pipe 131.

This apparatus comprises a second reservoir 137 which comprises the at least one main active product as defined above.

The second reservoir 137 is in fluidic communication with a second secondary inlet 138 of the main pipe 131. This second secondary inlet 138 is located downstream of the first secondary inlet 134.

The fluidic communication is carried out by means of a mixer 139.

Preferably, the mixer 139 operates with a suction system via the Venturi effect or via an injection pump.

This apparatus according to FIG. 4 also comprises a third reservoir 140 which comprises the at least one additive as defined above.

The third reservoir 140 being in fluidic communication with a third secondary inlet 141 of the main pipe 131. This third secondary inlet 141 is located downstream of the second secondary inlet 138.

The fluidic communication is carried out by means of a mixer 142.

Preferably, the mixer 142 operates with a suction system via the Venturi effect or via an injection pump.

The main pipe 141 is connected to at least one main nozzle 143. The number of main nozzles is not limiting in the apparatus according to the invention. However, this number is preferably between 1 and 12.

A grid 144 is located downstream of said at least one main nozzle 143.

Means configured for producing a blast 145 are located upstream of said at least one main nozzle 143 in order to produce a blast in the flow direction of the mixture present at the main inlet of the second module. In this FIG. 4, the means for producing a blast comprise a fan.

In order to operate the apparatus according to FIG. 4, the main inlet 132 is connected to an intake of main liquid. This main liquid then passes through the valve 136 in order to reach the first mixer 135. The main liquid is thus mixed with the at least one surfactant present in the first reservoir 133 and connected to the main pipe 131 by means of the first secondary inlet 134.

The mixture thus obtained arrives in the second mixer 139. The mixture is thus mixed with the at least one main active product present in the second reservoir 137 and connected to the main pipe 131 by means of the second secondary inlet 138.

The mixture thus obtained arrives in the third mixer 142. The mixture is mixed with the at least one additive present in the third reservoir 140 and connected to the main pipe 131 by means of the third secondary inlet 141.

The mixture thus obtained is sprayed by means of at least one main nozzle 143 onto a grid 144. Means configured for producing a blast 145 produce the bubbles of the foam by spraying gas into the mixture present on the grid 144.

FIG. 5 presents a second mode of implementation of the foam production apparatus according to the invention.

This apparatus takes up the elements from the apparatus according to FIG. 4. Consequently, the references of the common elements in FIG. 5 are the same as for FIG. 4 increased by 20.

This apparatus comprises the elements 151 to 165 which are arranged and which operate in the same way as above for the corresponding elements in FIG. 4.

This apparatus according to FIG. 5 also comprises a control system 166 controlling the operation of the valve 156, of the mixers 155, 159, 162 and of the means configured for producing a blast 165.

FIG. 6 presents a third mode of implementation of the foam production apparatus according to the invention.

This apparatus comprises a main pipe 171 having a main inlet 172. This main inlet is connected to an intake of main liquid. The intake of main liquid possibly being connected in the same way as described above.

The apparatus comprises a first reservoir 173 which comprises the at least one surfactant as defined above.

The first reservoir 173 is in fluidic communication with a first secondary inlet 174 of the main pipe 171.

The fluidic communication is carried out by means of a mixer 175.

Preferably, the mixer 175 operates with a suction system via the Venturi effect or via an injection pump.

Downstream of this first mixer 175, a valve 176 is present on the main pipe 171.

The main pipe 171 is connected to at least one main nozzle 177.

A grid 178 is located downstream of said at least one main nozzle 177.

Means configured for producing a blast 179 are located upstream of said at least one main nozzle 177 in order to produce a blast in the flow direction of the mixture present at the main inlet of the second module. In this FIG. 6, the means for producing a blast comprise a fan.

The second module also comprises an addition device for adding the at least one active product which comprises a supplementary reservoir 180 which comprises the at least one main active product, said supplementary reservoir 180 being connected to at least one supplementary nozzle 181, the flow of the at least one active product being in the same direction as the flow of the mixture present at the inlet of the second module.

In order to operate the apparatus according to FIG. 6, the main inlet 172 is connected to an intake of main liquid. This main liquid then passes through the valve 176 in order to reach the first mixer 175. The main liquid is thus mixed with the at least one surfactant present in the first reservoir 173 and connected to the main pipe 171 by means of the first secondary inlet 174.

The mixture thus obtained is sprayed by means of at least one main nozzle 177 onto a grid 178. Means configured for producing a blast 179 produce the bubbles of the foam by spraying gas into the mixture present on the grid 178.

Added to this mixture by means of the supplementary nozzle 181 is the at least one main active product stored in the supplementary reservoir 180.

FIG. 7 presents a fourth mode of implementation of the foam production apparatus according to the invention.

This apparatus comprises a main reservoir 193 which comprises the main liquid as defined above.

This reservoir 193 may in particular be under a pressure greater than the atmospheric pressure.

This reservoir 193 is connected to the inlet 192 of a main pipe 191.

The apparatus comprises a first reservoir 194 which comprises the at least one surfactant as defined above.

The first reservoir 194 is in fluidic communication with a first secondary inlet 195 of the main pipe 191.

The fluidic communication is carried out by means of a mixer 196.

Preferably, the mixer 196 operates with a suction system via the Venturi effect or via an injection pump.

Downstream of this first mixer 196, a valve 197 is present on the main pipe 191.

This apparatus comprises a second reservoir 198 which comprises the at least one main active product as defined above.

The second reservoir 198 is in fluidic communication with a second secondary inlet 199 of the main pipe 191. The second secondary inlet 199 is located downstream of the first secondary inlet 195.

The fluidic communication is carried out by means of a mixer 200.

Preferably, the mixer 200 operates with a suction system via the Venturi effect or via an injection pump.

This apparatus according to FIG. 7 also comprises a third reservoir 201 which comprises the at least one additive as defined above.

The third reservoir 201 being in fluidic communication with a third secondary inlet 202 of the main pipe 191. This third secondary inlet 202 is located downstream of the second secondary inlet 199.

The fluidic communication is carried out by means of a mixer 203.

Preferably, the mixer 203 operates with a suction system via the Venturi effect or via an injection pump.

The main pipe 191 is connected to a final reservoir 204. This final reservoir 204 comprises a sparging device 206. This sparging device is supplied by means of a reservoir 205 under pressure greater than the atmospheric pressure.

In order to operate the apparatus according to FIG. 7, the main reservoir 193 is filled with main liquid, everything being under a pressure greater than the atmospheric pressure.

The main liquid flows through the main inlet 192 of the main pipe 191. This main liquid then passes through the valve 197 in order to reach the first mixer 196. The main liquid is thus mixed with the at least one surfactant present in the first reservoir 194 and connected to the main pipe 191 by means of the first secondary inlet 195.

The mixture thus obtained arrives in the second mixer 200. The mixture is thus mixed with the at least one main active product present in the second reservoir 198 and connected to the main pipe 191 by means of the second secondary inlet 199.

The mixture thus obtained arrives in the third mixer 203. The mixture is mixed with the at least one additive present in the third reservoir 201 and connected to the main pipe 191 by means of the third secondary inlet 202.

The mixture thus obtained is stored in a final reservoir 204. In this final reservoir 204, a gas is made to bubble by means of a sparging system 206. The sparging system 206 makes it possible to produce the foam. This sparging system 206 is supplied by means of a reservoir 205 which comprises the gas under pressure greater than the atmospheric pressure.

FIG. 8 presents a fifth mode of implementation of the foam production apparatus according to the invention.

This apparatus comprises a main pipe 211. This main pipe is connected on the one hand to a single reservoir 212 which comprises the main liquid, the at least one surfactant and the at least main active product.

This single reservoir comprises an inlet 213 connected to a pyrotechnic system or to a compressed gas storage and/or production system 214.

This main pipe also comprises a valve 215 and is connected on the other hand to at least one main nozzle 216.

A grid 217 is located downstream of said at least one main nozzle 216.

The means configured for producing a blast 218 are located upstream of said at least one main nozzle 216 in order to produce a blast in the flow direction of the mixture present at the main inlet of the second module. In this FIG. 8, the means for producing a blast comprise a fan.

In order to operate the apparatus according to FIG. 8, the single reservoir 212 comprises a main mixture which comprises the main liquid, the at least one surfactant and the at least one main active product. This main mixture is driven towards the pipe 211 by means of the pyrotechnic system or the compressed gas storage and/or production system 214 connected to the inlet 213 of the single reservoir 212.

After having passed through the valve 215, the main mixture is sprayed by means of at least one main nozzle 216 onto a grid 217. Means configured for producing a blast 218 produce the bubbles of the foam by spraying gas into the mixture present on the grid 217.

1. An apparatus for producing foam comprising:
a first module (A) comprising a main outlet and delivering at said main outlet a mixture comprising a main liquid and at least one surfactant,
a second module (B) coupled to the main outlet of the first module (A) and producing a foam that comprises at least the main liquid, the at least one surfactant and at least one main active product, from the mixture delivered by the main outlet of the first module (A),
the mixture comprising the at least one main active product or
the second module (B) additionally comprising means configured so that the foam produced by the second module (B) comprises the at least one main active product, characterized in that said at least one main active product is selected from dyes, fluorescent products, chemical and/or biological markers, irritant products, gases, products that destroy the muscular and/or thought capacities of a living being and radar reflectors.

2. The apparatus as claimed in claim 1, characterized in that the foam produced by the apparatus has an expansion coefficient of greater than 10.

3. The apparatus as claimed in claim 1, characterized in that the mixture delivered by the main outlet of the first module comprises the at least one main active product and this first module comprises:
either a single reservoir (1) which comprises the main liquid, the at least one surfactant and the at least one main active product, this reservoir being connected to the main outlet (2) of the first module,
or a main reservoir (11) that comprises the main liquid, said main reservoir (11) being connected to a main inlet (13) of a main pipe (14), and a first reservoir (15) that comprises the at least one surfactant and the at least one main active product, said first reservoir (15) being in fluidic communication (16) with a first secondary inlet (17) of the main pipe (14), said main pipe (14) being connected to the main outlet (12) of the first module,
or a main reservoir (11) that comprises the main liquid and the at least one surfactant, said main reservoir (11) being connected to a main inlet (13) of a main pipe (14), and a first reservoir (15) that comprises the at least one main active product, said first reservoir (15) being in fluidic communication (16) with a first secondary inlet (17) of the main pipe (14), said main pipe (14) being connected to the main outlet (12) of the first module,
or a main reservoir (11) that comprises the main liquid and the at least one main active product, said main reservoir (11) being connected to a main inlet (13) of a main pipe (14), and a first reservoir (15) that comprises the at least one surfactant, said first reservoir (15) being in fluidic communication (16) with a first secondary inlet (17) of the main pipe (14), said main pipe (14) being connected to the main outlet (12) of the first module,
or a main reservoir (21) that comprises the main liquid, said main reservoir (21) being connected to a main inlet (23) of a main pipe (24), a first reservoir (25) that comprises the at least one surfactant, said first reservoir (25) being in fluidic communication (26) with a first secondary inlet (27) of the main pipe (24), and a second reservoir (28) that comprises at least one main active product, said second reservoir (28) being in fluidic communication (29) with a second secondary inlet (30) of the main pipe (24), said main pipe (24) being connected to the main outlet (22) of the first module,
or a main inlet (33) of a main pipe (34) connected to an intake of the main liquid, a first reservoir (35) which comprises the at least one surfactant and the at least one main active product, said first reservoir (35) being in fluidic communication (36) with a first secondary inlet (37) of the main pipe (34), said main pipe (34) being connected to the main outlet (32) of the first module,
or a main inlet (43) of a main pipe (44) connected to an intake of the main liquid, a first reservoir (45) which comprises the at least one surfactant, said first reservoir (45) being in fluidic communication (46) with a first secondary inlet (47) of the main pipe (44), and a second reservoir (48) which comprises the at least one main active product, said second reservoir (48) being in fluidic communication (49) with a second secondary inlet (50) of the main pipe (44), said main pipe (44) being connected to the main outlet (42) of the first module.

4. The apparatus as claimed in claim 1, characterized in that the second module also comprises means configured so that the foam produced by the second module comprises the at least one main active product and the first module comprises:
either a single reservoir (51) which comprises the main liquid, the at least one surfactant and optionally at least one secondary active product, this reservoir (51) being connected to the main outlet (52) of the first module,
or a main reservoir (61) which comprises the main liquid, and optionally at least one secondary active product, said main reservoir (61) being connected to a main inlet (63) of a main pipe (64), and a first reservoir (65) which comprises the at least one surfactant, said first reservoir (65) being in fluidic communication (66) with a first secondary inlet (67) of the main pipe (64), said main pipe (64) being connected to the main outlet (62) of the first module,
or a main reservoir (61) which comprises the main liquid, said main reservoir (61) being connected to a main inlet (63) of a main pipe (64), and a first reservoir (65) which comprises the at least one surfactant and optionally at least one secondary active product, said first reservoir (65) being in fluidic communication (66) with a first secondary inlet (67) of the main pipe (64), said main pipe (64) being connected to the main outlet (62) of the first module,
or a main reservoir (61) which comprises the main liquid and the at least one surfactant, said main reservoir (61) being connected to a main inlet (63) of a main pipe (64), and a first reservoir (65) which comprises at least one secondary active product, said first reservoir (65) being in fluidic communication (66) with a first secondary inlet (67) of the main pipe (64), said main pipe (64) being connected to the main outlet (62) of the first module,
or a main reservoir (71) which comprises the main liquid, said main reservoir (71) being connected to a main inlet (73) of a main pipe (74), a first reservoir (75) which comprises the at least one surfactant, said first reservoir (75) being in fluidic communication (76) with a first secondary inlet (77) of the main pipe (74), and a second reservoir (78) which comprises at least one secondary active product, said second reservoir (78) being in fluidic communication (79) with a second secondary inlet (80) of the main pipe (74), said main pipe (74) being connected to the main outlet (72) of the first module,
or a main inlet (83) of a main pipe (84) connected to an intake of the main liquid, a first reservoir (85) which comprises the at least one surfactant and optionally at least one secondary active product, said first reservoir (85) being in fluidic communication (86) with a first secondary inlet (87) of the main pipe (84), said main pipe (84) being connected to the main outlet (82) of the first module,
or a main inlet (93) of a main pipe (94) connected to an intake of the main liquid, a first reservoir (95) which comprises the at least one surfactant, said first reservoir (95) being in fluidic communication (96) with a first secondary inlet (97) of the main pipe (94), and a second reservoir (98) which comprises at least one secondary active product, said second reservoir (98) being in fluidic communication (99) with a second secondary inlet (100) of the main pipe (94), said main pipe (94) being connected to the main outlet (92) of the first module.

5. The apparatus as claimed in claim 1, characterized in that the second module comprises a main inlet (101, 111, 121) connected to the main outlet of the first module, said main inlet (101, 111, 121) being connected:
either to at least one main nozzle (102), a grid (103) being positioned downstream of said at least one main nozzle (102), and, upstream of said at least one main nozzle (102), means configured for producing a blast (104) in the flow direction of the mixture present at the main inlet (101, 111, 121) of the second module,
or to at least one main nozzle (112), a gas inlet area (114) in the form of a housing being positioned downstream of said at least one main nozzle (112), the longitudinal axis of this inlet area (114) being parallel or identical to the spraying axis of said at least one main nozzle (112), and downstream of this inlet area (114), a grid (113) positioned over the outlet of the inlet area (114),
or to a final reservoir (122), said final reservoir comprising a sparging system (123).

6. The apparatus as claimed in claim 5, characterized in that the means configured for producing a blast comprise a fan or a pyrotechnic system.

7. The apparatus as claimed in claim 2, characterized in that the single reservoir of the first module or the main reservoir of the first module have an inlet connected to a pyrotechnic system, or to a compressed gas storage and/or production system (214).

8. The apparatus as claimed in claim 2, characterized in that the single reservoir of the first module or the main reservoir of the first module are under a pressure greater than the atmospheric pressure.

9. The apparatus as claimed in claim 6, characterized in that the pyrotechnic system comprises an explosive composition.

10. The apparatus as claimed in claim 7, characterized in that the compressed gas storage and/or production system comprises a compressor and/or a pressurized gas cylinder.

11. The apparatus as claimed in claim 1, characterized in that the foam produced by the second module also comprises at least one additive.

12. The apparatus as claimed in claim 11, characterized in that the first module comprises at least one third reservoir (140, 160, 201) which comprises the at least one additive, said third reservoir (140, 160, 201) being in fluidic communication with a third secondary inlet (141, 161, 202) of the main pipe of the first module.

13. The apparatus as claimed in claim 11, characterized in that said at least one additive is selected from surfactants different from the at least one surfactant.

14. The apparatus as claimed in claim 11, characterized in that the mixture delivered at the main outlet of the first module has a content of additive that varies from 0.0001% to 10% by weight, relative to the total weight of the mixture delivered at the main outlet of the first module.

15. The apparatus as claimed in claim 2, characterized in that, when they are present, the fluidic communications of the reservoirs with the secondary inlets are carried out by means of mixers (135, 139, 142, 155, 159, 162, 175, 196, 200, 203).

16. The apparatus as claimed in claim 2, characterized in that the apparatus comprises a control system (166) that controls the operation, when they are present, of the valve (156) and/or of the mixers (155, 159, 162) and/or of the means configured for producing a blast (165).

17. The apparatus as claimed in claim 1, characterized in that the means configured so that the foam produced by the second module comprises the at least one main active product, comprise an addition device which comprises a supplementary reservoir (180) which comprises the at least one main active product, the supplementary reservoir (180) being connected to at least one means capable of adding the at least one main active product to the mixture present at the main inlet of the second module.

18. The apparatus as claimed in claim 17, characterized in that, when the main inlet of the second module is connected to at least one main nozzle, the means capable of adding the at least one main active product to the mixture present at the main inlet of the second module comprises at least one supplementary nozzle (181), the flow of the at least one main active product being in the same direction as the flow of the mixture present at the main inlet of the second module.

19. The apparatus as claimed in claim 1, characterized in that the main liquid is selected from a freshwater, a seawater, an oil, an alcohol and/or a mixture of these liquids.

20. The apparatus as claimed in claim 1, characterized in that said at least one surfactant is selected from protein-based surfactants obtained by hydrolysis of animal or plant proteins or synthetic-based surfactants derived from the chemical industry or else any molecule capable of generating a foam.

21. The apparatus as claimed in claim 1, characterized in that the content of surfactant in the mixture delivered at the main outlet of the first module varies from 1% to 10% by weight, relative to the total weight of the mixture present at the main outlet of the first module.

22. The apparatus as claimed in claim 1, characterized in that said at least one secondary active product is selected from dyes, fluorescent products, chemical and/or biological markers, irritant products, gases, products that destroy the muscular and/or thought capacities of a living being and radar reflectors.

23. The apparatus as claimed in claim 1, characterized in that said at least one main active product and/or said at least one optional secondary active product are selected from irritant products and products that destroy the muscular and/or thought capacities of a living being, more particularly said at least one main active product and/or said at least one optional secondary active product are capsaicin, capsicum oleoresin or a derivative thereof and nonivamide.

24. The apparatus as claimed in claim 1, characterized in that they are present in the mixture at the main outlet of the first module, the at least one main active product and the at least one optional secondary active product each have a content that varies from 0.1% to 10% by weight, relative to the total weight of the mixture delivered at the main outlet of the first module.

25. A foam capable of being produced by the apparatus as claimed in claim 1.

26. The foam as claimed in claim 25, characterized in that the main active product is located in the wall of the bubbles of the foam and/or in the form of gas inside the bubbles of the foam and/or in the form of fine liquid or solid particles in suspension inside the bubbles of the foam.

27. A motor vehicle, ship or aircraft comprising the apparatus as defined in claim 1.

28. A process for producing foam, comprising the following steps:
i) a first module that delivers at the main outlet a mixture comprising a main liquid and at least one surfactant, then
ii) a second module connected to the main outlet of the first module that produces a foam which comprises the main liquid, the at least one surfactant and at least one main active product, it being understood that the foam produced has an expansion coefficient of greater than 10 characterized in that said at least one main active product is selected from dyes, fluorescent products, chemical or biological markers, irritant products, gases, products that destroy the muscular or thought capacities of a living being and radar reflectors.
